(19) United States
(12) Patent Application Publication
(10) Pub. No.: US 2024/0077474 A1
(43) Pub. Date: Mar. 7, 2024

(54) SYSTEM AND METHODS FOR POSITIONING BIOMATERIAL ON A SUBSTRATE

(71) Applicant: Seagate Technology LLC, Fremont, CA (US)

(72) Inventors: James Froberg, Edina, MN (US); Gemma Mendonsa, Edina, MN (US); Calvin Daniel Nazareth, Minneapolis, MN (US)

(73) Assignee: Seagate Technology LLC, Fremont, CA (US)

(21) Appl. No.: 17/929,779

(22) Filed: Sep. 6, 2022

(51) Int. Cl. G01N 33/531; G01N 33/543

(52) U.S. Cl. CPC G01N 33/531; G01N 33/5436

(57) ABSTRACT

The present disclosure describes a system and methods for positioning a configured number of biomaterials on a surface. The method disclosed includes the formation of a membrane mask structure including a configured number of nanopores. The nanopores are generally sized to be the same size as, or slightly larger than a single target biomaterial. The masking membrane is then adhered to a substrate. The substrate may include a raw substrate, or may be functionalized for the purpose of binding biomaterials with greater attraction. A solution including the target biomaterials is then exposed to the masking membrane. One biomaterial is able to adhere to the substrate through each nanopore. The solution is rinsed from the membrane surface leaving only the biomaterial that has adhered to the substrate with a relatively strong binding force. The masking membrane may then either remain or may be removed.

### TECHNICAL FIELD

This disclosure relates to a method for positioning a biomaterial and immobilizing it on a substrate.

### BACKGROUND

Biomaterial positioning and immobilization is needed extensively in molecular biology research and the medical field. A number of techniques have arisen to accomplish biomaterial immobilization. These techniques typically include functionalizing the surface upon which the biomaterials are to be adhered to and performing some sort of conjugational chemistry to bind the biomaterials. While such techniques are entirely sufficient to mass-immobilize biomaterials over a surface, the ability to selectively position the biomaterial(s) and configure the number of biomaterials to be immobilized is far more difficult. This is important because a number of applications require the positioning of a single biomaterial on a surface. Biomaterials may include cell fragments, proteins, or other biomolecules.

Particularly, biosensing applications, such as DNA sequencing, disease detection, drug development and personal health monitoring systems require single protein positioned on a surface of a substrate or sensor. Existing techniques do exist for single protein positioning, but these methodologies generally require incubation of droplets with large numbers of the protein on the sensor surface while configuring the incubation time and protein concentrations until an exact combination of factors are reached to result in a single protein binding as desired. These techniques, as one may imagine, have low yields due to the fact that multiple protein bindings are common. For this reason, improvements in positioning configurable numbers of proteins on a surface with higher yields and processing throughput are desirable.

### SUMMARY

The present disclosure describes a system and methods for positioning a configured number of biomaterials on a surface. Such systems and methods provide significant yield improvements over known single-biomaterial immobilization techniques. Single biomaterial conjugated surfaces have significant implications for many biosensing applications. Biomaterials may include proteins, cell fragments, or other biomolecules (e.g., DNA strand and the like).

In particular, the method disclosed includes the formation of a membrane mask structure including a configured number of nanopores. The nanopores are generally sized to be the same size as, or slightly larger than a single target biomaterial. In some cases, the nanopore(s) are formed by Controlled Breakdown (CBD), but it may also be possible to form the nanopores using lithographic techniques.

In some embodiments, the membrane includes a dielectric material, and has a thickness of approximately 2-20 nm. Given biomaterial sizes, the nanopore is generally between 2-50 nm.

The masking membrane is then adhered to a substrate. Possible adhesion methods could involve utilizing Van Der Waals interactions, ultraviolet curing, or pi-pi stacking. The substrate may include a sensor device, in some embodiments. In some particular embodiments, the substrate may include a graphene sensor. The substrate may include a raw substrate, or may be functionalized for the purpose of binding biomaterials with greater attraction. For example, an active layer may be added to the substrate. The active layer could include any of linker molecules, altered hydrophobicity, metallic deposition, or some combination thereof.

A solution including the target biomaterials is then exposed to the masking membrane. One biomaterial is able to adhere to the substrate through each nanopore. In some cases, where only one nanopore is present, this means a single biomaterial is adhered to the substrate. Only one biomaterial adheres per nanopore because, physically, there is only enough space for a single biomaterial to ‘fit’ per nanopore. Biomaterials adhere very poorly to the masking membrane, thus, when the solution is rinsed from the membrane surface, only the biomaterial that has adhered to the substrate with a relatively strong binding force, remains. The masking membrane may then either remain or may be removed. Removal may include chemical polishing of the mask with a solvent that preferentially dissolves the membrane, or through the physical removal of the mask. Regardless, the result of the process is to leave a functional, immobilized biomaterial conjugated to the substrate surface, at a configured position.

Note that the various features of the present invention described above may be practiced alone or in combination. These and other features of the present invention will be described in more detail below in the detailed description of the invention and in conjunction with the following figures.

### BRIEF DESCRIPTION OF THE DRAWINGS

In order that the present invention may be more clearly ascertained, some embodiments will now be described, by way of example, with reference to the accompanying drawings, in which:

FIG. 1A is an illustration of a substrate layer for the positioning of a single biomaterial, in accordance with aspects of this disclosure.

FIG. 1B is an illustration of a functionalized substrate for the positioning of a single biomaterial, in accordance with aspects of this disclosure.

FIG. 1C is an illustration of a functionalized substrate layer with a raw masking membrane for the positioning of a single biomaterial, in accordance with aspects of this disclosure.

FIG. 1D is an illustration of a functionalized substrate layer with a processed masking membrane for the positioning of a single biomaterial, in accordance with aspects of this disclosure.

FIG. 1E is an illustration of a processed masking membrane adhered to a functionalized substrate layer for the positioning of a single biomaterial, in accordance with aspects of this disclosure.

FIG. 1F is an illustration of a processed masking membrane adhered to a functionalized substrate layer and exposed to a biomaterial solution for the positioning of a single biomaterial, in accordance with aspects of this disclosure.

FIG. 1G is an illustration of a processed masking membrane adhered to a functionalized substrate layer and with the biomaterial solution rinsed off to leave a single adhered biomaterial, in accordance with aspects of this disclosure.

FIG. 1H is an illustration of a functionalized substrate layer with the processed masking membrane removed to leave a single biomaterial positioned upon the substrate, in accordance with aspects of this disclosure.

FIG. 2 is an illustration of an example graphene sensor apparatus with a single polymerase biomaterial adhered to the sensor stack for sequencing a single stranded DNA segment, as an example of a use case for the disclosed method of adhering a single protein to the substate, in accordance with aspects of this disclosure.

FIG. 3 is a flow diagram of an example process for the positioning of a single protein upon a substrate, in accordance with aspects of this disclosure.

FIG. 4 is a flow diagram for an example process of substrate preparation, in accordance with aspects of this disclosure.

FIG. 5 is a flow diagram of an example process for mask membrane preparation, in accordance with aspects of this disclosure.

FIG. 6 is a flow diagram of an example process for protein deposition, in accordance with aspects of this disclosure.

### DETAILED DESCRIPTION

The present invention will now be described in detail with reference to several embodiments thereof as illustrated in the accompanying drawings. In the following description, numerous specific details are set forth in order to provide a thorough understanding of embodiments of the present invention. It will be apparent, however, to one skilled in the art, that embodiments may be practiced without some or all of these specific details. In other instances, well known process steps and/or structures have not been described in detail in order to not unnecessarily obscure the present invention. The features and advantages of embodiments may be better understood with reference to the drawings and discussions that follow.

Aspects, features and advantages of exemplary embodiments of the present invention will become better understood with regard to the following description in connection with the accompanying drawing(s). It should be apparent to those skilled in the art that the described embodiments of the present invention provided herein are illustrative only and not limiting, having been presented by way of example only. All features disclosed in this description may be replaced by alternative features serving the same or similar purpose, unless expressly stated otherwise. Therefore, numerous other embodiments of the modifications thereof are contemplated as falling within the scope of the present invention as defined herein and equivalents thereto. Hence, use of absolute and/or sequential terms, such as, for example, “will,” “will not,” “shall,” “shall not,” “must,” “must not,” “first,” “initially,” “next,” “subsequently,” “before,” “after,” “lastly,” and “finally,” are not meant to limit the scope of the present invention as the embodiments disclosed herein are merely exemplary.

The present invention relates to the immobilization and positioning of at least one biomaterial upon a substrate layer in a configurable manner. In particular, it is possible using the disclosed systems and methods, to adhere a single protein to a substrate layer, with a high throughput, and relatively high yields. This is important because a number of applications require the positioning of a single biomaterial on a surface. Particularly, biosensing applications, such as DNA sequencing, disease detection, drug development and personal health monitoring systems require single protein positioned on a surface of a substrate or sensor.

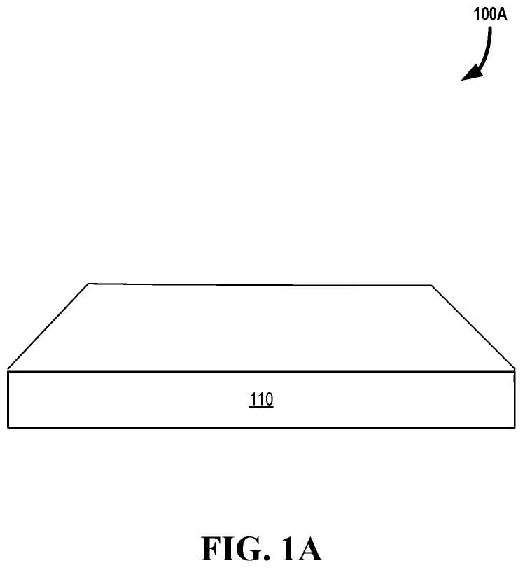

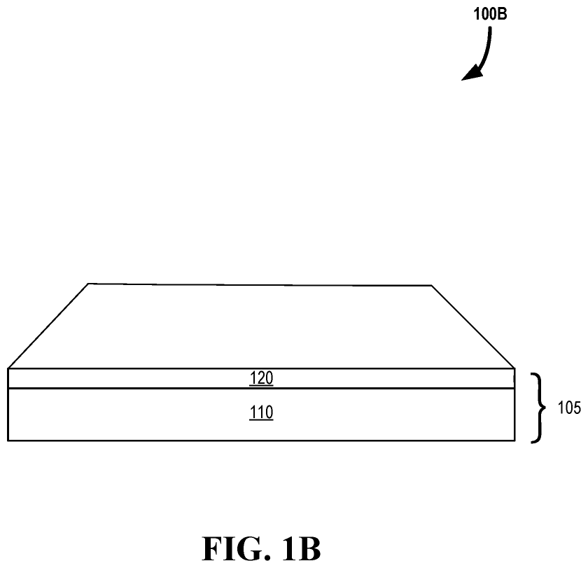

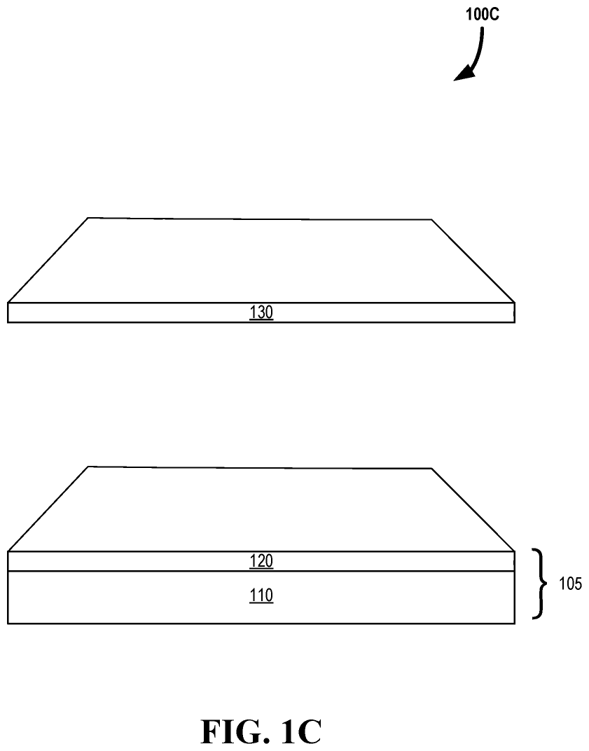

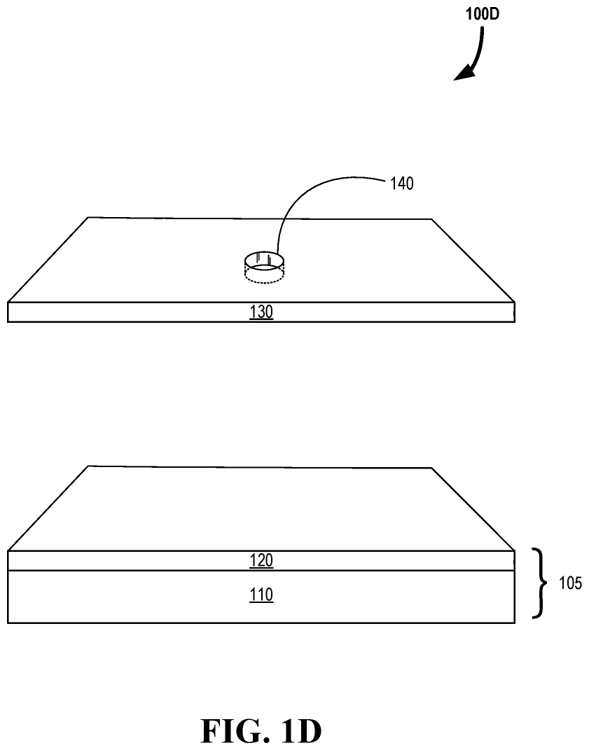

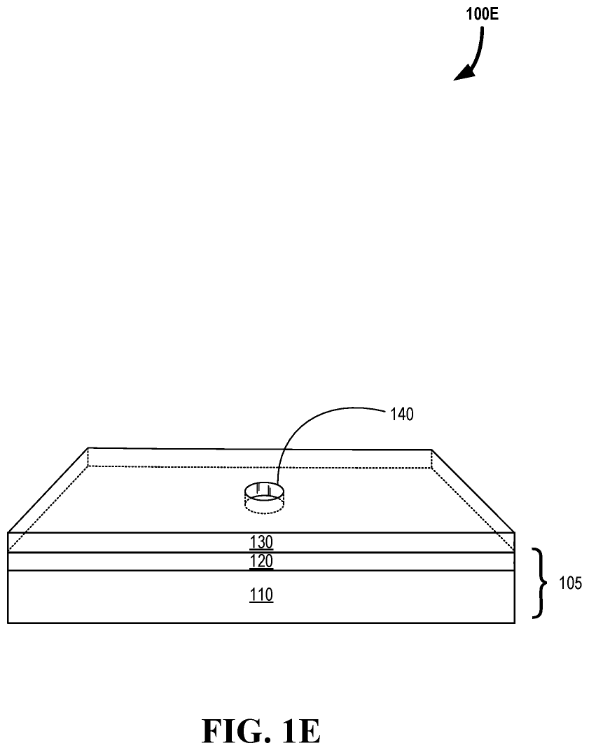

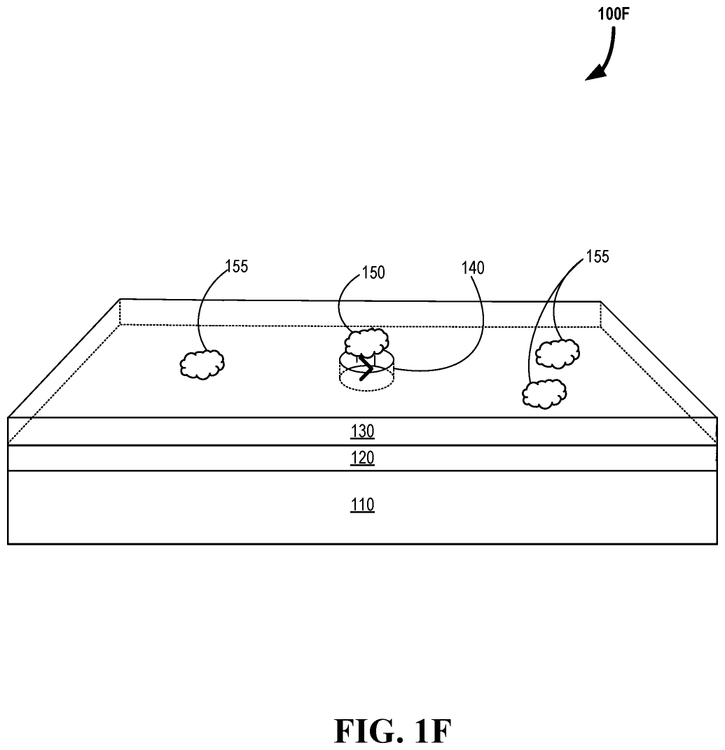

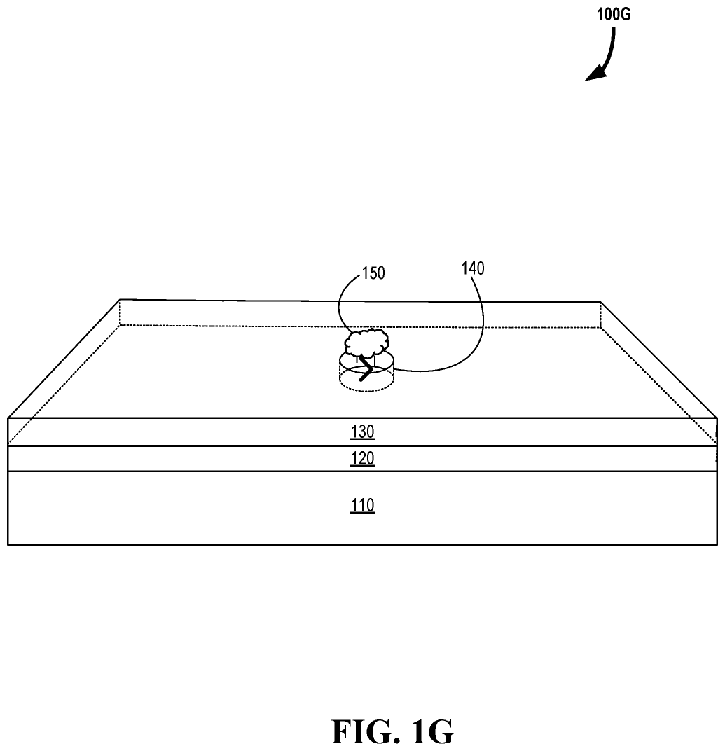

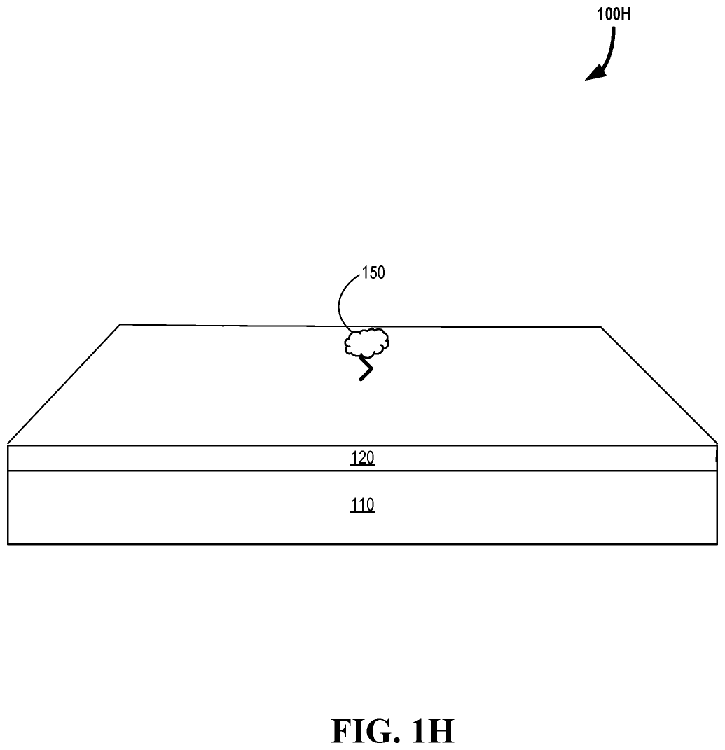

To facilitate discussion, FIG. 1A provides an illustration, shown generally at 100A, of a raw substrate layer 110 for the positioning on a single biomaterial upon. For the purposes of this disclosure the term “substrate” is employed as a generic term for the underlying structure that the biomaterial will ultimately be adhered to (both functionalized, and in its raw form). Likewise, “biomaterial” may refer to a protein molecule, a cell fragment, exosome, or any biological molecule or molecule collections. Often the raw substrate 110 may include a sensor device, such as a graphene sensor, giant magnetoresistance (GMR) sensor, optical or fluorescence based sensor, MRI, X-ray sensor, photonic sensor, electrical sensor, or the like. In other embodiments, the substrate may include a glass slide, silicon wafer, polymer structure, metallic surface, waveguide, microfluidic channel, or virtually any surface that is relatively flat. Regardless, for the purpose of this disclosure, the term ‘raw substrate’ is intended to refer to any possible surface that the positioning of a biomaterial is desired.

In some embodiments, the raw substrate 110 may possess properties that are conducive to the direct binding of proteins or other biomaterials such as exosomes or cell fragments. For example, a hydrophobic surface could be used to attract hydrophobic ends of biomaterials, or a low voltage applied to the surface could be used to attract biomaterials with the complimentary charge. Proteins tend to adhere well to plastic surfaces. However, in most situations the raw substrate 110 itself does not possess the needed properties to bind strongly enough to the biomaterial, and as such the raw substrate 110 is required to be functionalized. FIG. 1B provides an illustration, shown generally at 100B, of such a functionalized substrate 105, which includes an active layer 120 deposited upon the surface of the raw substrate 110. In some embodiments, the active layer 120 may include a single layer; however, in alternate embodiments, the active layer 120 may comprise two or more discrete layers. For example, a common approach to biomaterial binding is to leverage ligands to adhere to the biomaterial. However, the ligands themselves do not adhere well to most underlying substrates. As such, it is possible to layer the raw substrate 110 first with gold (or other metallic surface) to which the ligands preferentially couple to. The depositing of the gold upon the substrate may be performed via techniques such as sputtering, spin-coating or a photolithography process, vapor deposition, or electroplating. In some embodiments, the metallic layer may be approximately 5-20 nm thick and may consist of gold (Au) or Cobalt (Co). Covering the metallic layer is the ligand layer which functionalizes the metallic layer. In some embodiments, Au—S bonding may be employed to couple the ligands of the ligand layer to the metallic layer. Another alternative is to attach ligands directly to the raw substrate 110 without a metallic layer between. For example, binding ligands may be attached to the raw substrate 110 surface via a pyrene foot and pi-pi stacking.

Regardless, the active layer 120 coupled to the surface of the raw substrate 110 may be referred to as a ‘functionalized substrate’ or an ‘active substrate’ interchangeably. Occasionally, the term ‘substrate’, without a qualifier, may be used to refer to the active substrate 105. In some embodiments, where the raw substrate 110 possesses the needed properties to bind to biomaterials, the raw substrate 110 itself may be referred to as an active substrate 105 or a functionalized substrate 105.

In FIG. 1C, an illustration is presented, shown generally at 100C, of the functionalized substrate 105 with a masking membrane 130 present. In some references the masking membrane 130 may be referred to simply as the ‘mask’ or conversely the ‘membrane’. Regardless of terminology employed, the masking membrane may be comprised of a dielectric material. In some embodiments, the masking membrane 130 is comprised of silicon-nitride, hafnium-oxide, or a polymer material (e.g., polymethyl methacrylate, polyethylene glycol, lipid bilayer membrane). In some particular embodiments, the thickness of the masking membrane 130 may vary between about 2-20 nm.

It should be noted that the terms “about”, “approximately”, “roughly” and the like may be employed. In general, these terms are intended to indicate that the value or range provided may vary by a set amount. Generally, the term “about” is intended to suggest a variation of plus or minus ten percent of the value provided. The terms “approximately” or “roughly” may indicate a value that is within one standard deviation of the provided value or range.

It should further be noted that the masking membrane 130 is illustrated here substantially parallel to the surface of the functionalized substrate 105, yet physically removed from the functionalized substrate 105. This is entirely for the purposes of providing clarity of the various elements. In some embodiments, this masking membrane 130 is adhered to, or placed adjacent to, the functionalized substrate 105, even before the nanopore formation (covered in greater detail below).

In some embodiments, the masking membrane 130 may be formed independently from the functionalized substrate 105. For example, the masking membrane 130 may be generated using spin-coating, sputtering or photolithography techniques on a separate chip if the process would damage the active substrate, and then transferred onto the active substrate after creation. Other ways of generating the membrane may include Self-assembly or photopolymerization of a thin-film. In alternate embodiments, the membrane may be formed directly upon the functionalized substrate 105 if the substrate would not be damaged by the previously mentioned processes for membrane creation.

Turning to FIG. 1D, an illustration, shown generally at 100D, is provided wherein the masking membrane 130 has a nanopore 140 inserted in it. As with the prior figure, this illustration shows the masking membrane 130, with attendant nanopore 140, as being physically separated from the functionalized substrate 105. This is again, purely for clarity purposes, and the masking membrane 130 may be adjacent to the functionalized substrate 105. In some embodiments, however, it is advantageous to generate the masking membrane 130 with attendant nanopore 140 independently from the functionalized substrate 105, and only after the processed masking membrane 130 with a nanopore 140 is generated, is it adhered to the functionalized substrate 105.

In some embodiments, the nanopore 140 is generated using Controlled Breakdown (CBD) methodology. In this technique a voltage is applied across a thin membrane and then slowly increased until an electric discharge across the membrane breaks a hole through it. This is often accomplished by positioning the membrane between two chambers containing electrolyte solutions and using the electrolyte solutions to apply a voltage across the membrane. In other embodiments, the nanopore 140 may be generated via other destructive techniques, such as poking a hole through the membrane with an atomic force microscopy tip, using a laser to break a hole through the membrane, or even alternative CBD methods where metal electrodes could replace one or both of the electrolyte solutions, or via electroporation, induction of transmembrane channel proteins, or via chemical means.

It should be noted that the present illustration (and subsequent illustrations) shows a masking membrane 130 with a single nanopore 140 in the center of it. This may be preferable when a single biomaterial is desired to be positioned in the middle of the substrate—such as when a biomaterial is immobilized to a sensor device. However, it is entirely possible that more than one nanopore 140 may be present within the masking membrane 130 if multiple biomaterials are to be positioned upon the functionalized substrate 105.

Generally, the nanopore 140 diameter is configurable from between about 2-50 nm in diameter. This configurable diameter is generally sized to be approximately the width of the biomaterial that is intended to be immobilized. In some cases, the nanopore 140 diameter may be configured to be larger than the diameter of a single biomaterial, but less than the diameter of two biomaterials. This ensures that a single biomaterial of interest is able to conjugate to the functionalized substrate 105 without a second biomaterial likewise attaching, as will be discussed in further detail below.

In FIG. 1E an illustration, shown generally at 100E, of the masking membrane 130 with a nanopore 140 present is placed adjacent to the functionalized substrate 105. This placement is proximal to, and parallel with the functionalized substrate's 105 surface. In some embodiments, the masking membrane 130 is adhered to the functionalized substrate 105 as to prevent biomaterials from inadvertently ‘slipping under’ the masking membrane 130 during biomaterial application. In some embodiments, the masking membrane 130 is adhered to the functionalized substrate 105 using Van Der Waals interactions, or ultraviolet curing.?

After the masking membrane 130, with attendant nanopore(s) 140, is coupled to the functionalized substrate 105, the biomaterial application step is performed. FIG. 1F is an illustration, shown generally at 100 F, of the biomaterial positioning structure during the biomaterial application. During this process, a solution of biomaterials is applied to the masking membrane's 130 surface. These biomaterials 155 coat the masking membrane, except for a single biomaterial 150 which happens to couple directly to the active layer 120 of the functionalized substrate 105 through the opening caused by the nanopore 140.

Conjugation of the biomaterial 150 to the active layer may include Bioconjugation via ligand/protein interactions, Click chemistry, or carbodiimide chemistry like the EDC/NHS reaction In some embodiments, the active layer 120 includes ligands. The ligand, in some embodiments, may include a COOH group that is then reacted with the biomaterial 150 via a NH 2 group located on the biomaterial 150 surface using NHS/EDC reaction. Such a coupling technique generally avoids impacting the carboxyl groups of the biomaterial 150. In NHS/EDC reactions, the EDC (a water soluble cross-linker agent) forms amine reactive O-acylisourea intermediate that spontaneously reacts with primary amines to form an amine bond. This process binds the biomaterial 150 relatively strongly to the active layer 120 surface.

It should be noted that a single biomaterial will attached in this manner for every nanopore 140 present in the masking membrane 130. As such, a configurable number of biomaterials may be immobilized upon the functionalized substrate 105 in configured positions. As noted before, only a single biomaterial 150 is capable of being adhered to the functionalized substrate 105 through a given nanopore 140 due to the configured size of the nanopore 140. Because the nanopore 140 is substantially the same size as the biomaterial 150 in diameter, or larger than the biomaterial 150 but smaller than two biomaterials, there is a physical size limitation to more than one biomaterial being able to bind to the functionalized substrate 105.

FIG. 1G provides an example illustration, shown generally at 100G, of the single biomaterial 150 bound to the functionalized substrate 105 through the nanopore 140. In this example illustration, the masking membrane 130 has been rinsed with a solution not containing any biomaterials. In some embodiments, the rinsing solution may be water, a biomaterial buffer solution, a soap solution, or any other suitable rinse. As the biomaterials 155 were only weakly bound to the masking membrane's 130 surface (or not bound at all), these biomaterials 155 are removed by the rinsing step. Only the more strongly bound biomaterial 150 which is adhered to the functionalized substrate 105 remains bound through the rinsing process.

FIG. 1H provides an example illustration, shown generally at 100H, of the biomaterial positioned structure with the masking membrane 130 having been removed. This removal may be performed by chemical removal or physical removal of the masking membrane. In some embodiments, the removal of the masking membrane 130 may enable the skipping of the rinsing step (as the excess biomaterials 155 are removed along with the membrane).

It should be noted that the system may also be leveraged, in accordance with some embodiments, with the masking membrane still attached. In these situations, the masking membrane 130 removal process is not completed, and rather the structure found in FIG. 1G may be utilized for downstream processes. Removal of the masking membrane 130 may be determined by the type of downstream processing that is being performed, characteristics of the bound biomaterial 150, length of the binding ligands found in the active layer 120, or if the mask being there somehow inhibits the attached biomaterial's ability to function in the intended way. Or alternatively, the mask could be left in place if it does not inhibit the biomaterial's process or if the mask removal process poses a risk of damaging or removing the attached biomaterial.

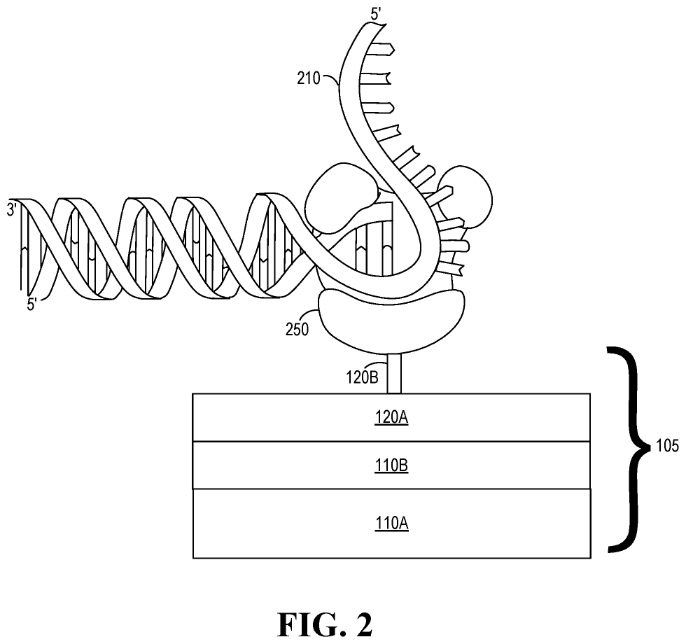

After the positioned biomaterial structure is thus generated, the device may be employed in downstream processing. This processing may include a myriad of different biosensing and nano-sensing techniques, as well as drug discovery. The biomaterial 150 selected for binding largely is dictated by the downstream application. For example, one form of downstream processing may include the sensing of DNA sequences determined through conjugation of labeled base pairs with a single stranded DNA (ssDNA) through a polymerase protein (e.g., sequencing by synthesis). FIG. 2 is an illustration of an example graphene sensor apparatus 170 performing such a sequencing process. The ssDNA segment 210 may be generated by denaturing a segment of double helical DNA through elevated temperatures or via chemical means. Generally, raising the temperature of a DNA containing solution to approximately 90 degrees Celsius or above causes the DNA strands to separate from one another to generate ssDNA segments 210. The addition of particular chemical reagents may lower the temperatures needed to form the ssDNA segments 210. Such reagents may include compounds such as urea and formamide, for example. Additionally, enzymes known as helicase may be leveraged to generate ssDNA segments 210.

DNA consists of a phosphate backbone and a series of base pairs (BPs) or nucleoside triphosphates. These BPs consist of adenine (A), guanine (G), cytosine (C) and thymine (T). When in a double helical configuration adenine binds to thymine, and cytosine binds to guanine. The replication of DNA involves the unwinding of the double helical (duplex) structure, separation of the two strands into ssDNA segments 210, and the processing of the ssDNA segment 210 through a DNA polymerase molecule 250, which in this case is the specific biomaterial 150 that has been positioned upon the functionalized substrate 105.

The DNA polymerase 250 is a class of polymerase molecules. Generally, the class of polymerases are highly conserved between individual molecules, and as such, no particular DNA polymerase 250 is required to be leveraged by any given aspect of this disclosure. In general, however, it may be desirous to utilize a DNA polymerase 250 that include a “proofreading” ability. This ability allows the DNA polymerase 250 molecule to detect when an incorrect BP has been introduced, and then enables the DNA polymerase 250 to move backwards (from the 5′ to the 3′ direction) along the ssDNA segment 210 to excise the incorrect BP (such errors occur approximately once every one billion BPs that are copied). After moving backwards and removing the incorrect BP, the DNA polymerase 250 may continue forward along the 5′ direction incorporating BPs along the ssDNA to form a full double helical DNA strand.

In some aspects of this disclosure, the DNA polymerase 250 is bound to a graphene sensor apparatus 110B by a binding ligand 120B or other suitable chemical structure. The ligand 120B, in some embodiments, may include a COOH group that is then reacted with the DNA polymerase 250 via a NH 2 group located on the DNA polymerase 250 surface using NHS/EDC reaction. Such a coupling technique generally avoids impacting the carboxyl groups of the DNA polymerase 250. In NHS/EDC reactions, the EDC (a water soluble cross-linker agent) forms amine reactive O-acylisourea intermediate that spontaneously reacts with primary amines to form an amine bond. By anchoring the DNA polymerase 250 to the graphene sensor apparatus, the molecule is maintained at a configurable distance from the sensor surface (based upon the binding ligand 120B length).

In some aspects of the disclosure, only a single DNA polymerase 250 may be immobilized upon the graphene sensor apparatus. This may be performed by using a membrane with a single nanopore as a mask that only allows a single DNA polymerase 250 molecule to be deposited upon the ligand surface 120B, as outlined in considerable detail above.

The graphene sensor apparatus itself includes a graphene sensor 110B, which together with a supporting structure 110A forms the raw substrate 110 of FIGS. 1A-1G. The supporting structure 110A may be any structure that provides mechanical stability to the raw substate 110. The surface of the graphene sensor 110B may be covered by a metallic layer 120A. The metallic layer 120A may be approximately 5-20 nm thick and may consist of gold (Au) or Cobalt (Co), in some aspects of the present disclosure. Covering the metallic layer 120A is a ligand layer 120B which functionalizes the metallic layer 120A. The metallic layer 120A and ligand layer 120B together form the active layer 120 of the functionalized substate 105. In some embodiments, Au—S bonding may be employed to couple the ligands of the ligand layer 120B to the metallic layer 120A. Another alternative is to attach ligands directly to the graphene layer 120B without a metallic layer between. For example, binding ligands may be attached to the graphene layer via a pyrene foot and pi-pi stacking.

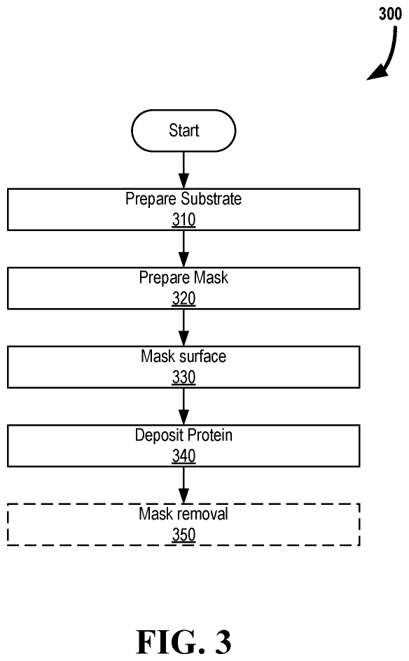

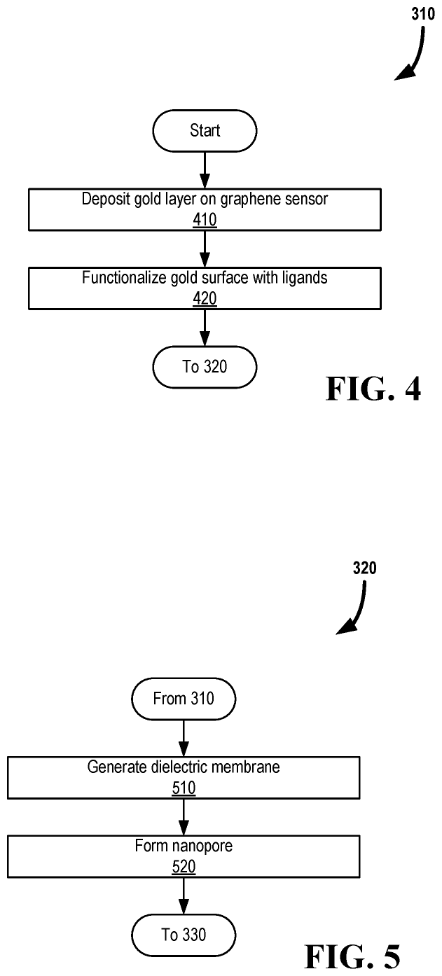

Turning now to the processes involved for the positioning of a single biomaterial 150 on a functionalized substrate 105 is FIG. 3, shown generally at 300. In this example process, the functionalized substrate 105 is first prepared (at 310). FIG. 4 provides an example of this sub-process in greater detail. Initially, a raw substrate 110 is procured or manufactured. As noted previously, the raw substrate 110 is often a sensor device, but may also include other substrates such as a glass slide (or other RF transparent substrate for imaging), or the like. In some particular embodiments, the raw substate 110 may include a graphene or giant magneto resistive (GMR) sensor. Generally, such raw substrates 110 include a structural layer upon which the sensor elements are deposited.

Regardless of raw substrate type, the device often requires functionalization through the application of an active layer 120 in order to bind proteins more securely. It is possible, however, that the nature of the raw substate 110 is such that it adheres preferentially to biomaterials without being functionalized. In such cases, the steps listed in FIG. 4 are not required. However, for most raw substrates 110 there is a need to prepare the substrate before further processing.

Functionalization of the raw substrate 110 may take many forms. One method of functionalizing the substrate is to alter the surface chemistry of the raw substrate (e.g., altering the hydrophobicity, charge or the like). Another methodology, which is exemplified in this FIG. 4, is to coat the raw substrate (at 410) with a metallic layer (e.g., gold or copper for example). This metallic layer may enable the binding of a ligand layer (at 420) to the raw substate 110. The result of the ligand coated raw substrate is the functionalized substrate 105 which is capable of adhering biomaterials to the ligand(s) through combinational chemistry, without disrupting the biomaterial's functionality.

Returning to FIG. 3, after generating the functionalized substrate 105, the masking membrane 130 may be generated (at 320). FIG. 5 provides a more detailed flow diagram of the sub-process of masking membrane 130 formation. The membrane is first procured or manufactured. In some cases, this may involve deposition of the membrane material directly upon the active layer 120 of the functionalized substrate 105. For example, membrane growth, vapor deposition or sputtering techniques may be employed for layering the membrane. However, in some particular embodiments, the masking membrane 130 may instead be prepared independently from the functionalized substrate 105 and subsequently added to it. In these situations, a dielectric membrane may be initially generated (at 510). In some cases, the membrane may be comprised of silicon-nitride, hafnium-oxide or a polymer material such as polymethyl methacrylate, polyethylene glycol, lipid bilayer membrane, and have a thickness of between about 2-20 nm. After membrane generation one or more nanopores may be formed within the membrane (at 520). Nanopore formation may include lithography techniques for destructively etching the pore out from the membrane, or in some particular situations may include formation using CBD methods where a voltage is applied across a dielectric membrane and then slowly increases until dielectric breakdown occurs and an electric discharge is sent through the membrane, boring a nanopore through it as this happens. This is similar to what causes lightning to form, but since air is a gas, it can fill the path bored through it by the lightning afterwards, but in a solid material like the membrane, the hole is left unfilled once the electric discharge is done. Nanopores are generally 2-50 nm in diameter, and are sized, configurably, to substantially match or exceed the size of the target biomaterial (yet remain smaller than two biomaterials in size).

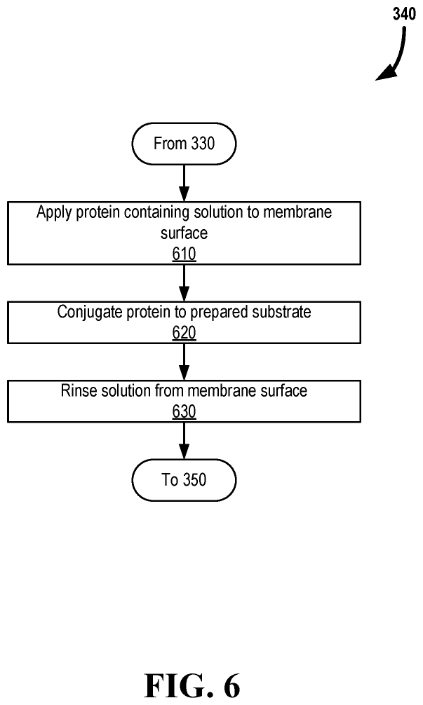

Returning to FIG. 3, after masking membrane 130 formation, the membrane may be placed (at 330) adjacent to, and substantially parallel with, the active layer 120 of the functionalized substrate 105. In some cases, the masking membrane 130 is adhered to the functionalized substrate 105 via linker molecules, altered hydrophobicity, metallic deposition, or some combination thereof. After the functionalized substrate 105 has been masked in this manner, the biomaterial may be deposited (at 340) upon the structure. FIG. 6 provides a more detailed description of an example of this biomaterial deposition sub-process. Initially many copies of the target biomaterial are suspended in a solution. This solution generally includes buffering agents and the like. Solution conditions (e.g., pH, molarity, etc.) are generally optimized for the binding of the biomaterials to ligands. The solution is then applied to the masking membrane 130 surface (at 610). The solution is incubated for an optimized time period at an optimized temperature in order to conjugate the biomaterial to the ligand layer of the functionalized substrate (at 620). After biomaterial binding through the one or more configured nanopores is complete, the solution may be rinsed from the membrane surface (at 630). Solution rinsing may be by water, a surfactant solution, a protein buffer solution, or the like.

Returning to FIG. 3, after the biomaterial has been bound though the nanopore, as outlined above, it is possible to remove the masking membrane (at 350). Depending upon downstream usage of the positioned biomaterial structure, it may instead be desirous to leave the masking membrane in place. As such, step 350 may be optional, as indicated by the dashed nature of this process step. If the masking membrane is to be removed, it is further possible to skip the membrane rinsing step of FIG. 6, as the excess biomaterials will be removed with the membrane. Membrane removal may include either physical or chemical removal of the membrane.

While this invention has been described in terms of several embodiments, there are alterations, modifications, permutations, and substitute equivalents, which fall within the scope of this invention. It should also be noted that there are many alternative ways of implementing the methods and apparatuses of the present invention. It is therefore intended that the following appended claims be interpreted as including all such alterations, modifications, permutations, and substitute equivalents as fall within the true spirit and scope of the present invention.

What is claimed is:

1. A method for selectively positioning a biomaterial molecule on a substrate comprising:
adhering a mask upon an active substrate, wherein the mask includes one or more nanopores of configured size; and
conjugating at least one biomaterial to the active substrate within the one or more nanopores.

2. The method of claim 1, further comprising exposing a solution with the biomaterial to the masked active substrate.

3. The method of claim 2, further comprising rinsing the solution off the mask.

4. The method of claim 3, further comprising removing the mask after conjugating.

5. The method of claim 1, wherein the at least nanopore is selectively positioned.

6. The method of claim 5, further comprising depositing an initial mask on the active substrate and generating the at least one nanopores in the initial mask.

7. The method of claim 6, wherein the generating the at least one nanopores utilized lithography techniques.

8. The method of claim 1, wherein the mask is a dielectric membrane with a thickness of about 2-20 nm.

9. The method of claim 8, wherein the at least one nanopores are generated within the dielectric membrane using Controlled Breakdown (CBD) methodology.

10. The method of claim 1, wherein the at least one nanopores are between about 2-50 nm in diameter.

11. The method of claim 1, wherein the at least one nanopore is about the same diameter as the biomaterial.

12. The method of claim 1, wherein the at least one nanopore is larger in diameter as the biomaterial and smaller in diameter as two biomaterials.

13. The method of claim 1, wherein the active substrate is a sensor covered in an active layer.

14. The method of claim 13, wherein the sensor is a graphene sensor.

15. The method of claim 13, wherein the active layer includes at least one of linker molecules, altered hydrophobicity, metallic deposition, or some combination thereof.

16. The method of claim 1, wherein the adhering the mask includes at least one of Van Der Waals interaction, ultraviolet curing, and pi-pi stacking.

17. A biomaterial positioning structure comprising:
a substrate, wherein the substrate includes an active layer; and
a mask adhered to the active layer of the substrate, wherein the mask includes a first surface and a second surface substantially parallel to one another, and wherein at least one nanopore is selectively positioned within the mask, wherein the nanopore includes an aperture which extends from the first surface to the second surface, wherein the aperture of the at least one nanopore is between about 2-50 nm in diameter, and wherein the distance between the first surface and the second surface is about between 2-20 nm.

18. The biomaterial positioning structure of claim 17, wherein the substrate includes a sensor.

19. The biomaterial positioning structure of claim 17, wherein mask is a dielectric membrane and the at least one nanopore is generated by Controlled Breakdown (CBD) methodology.

20. The biomaterial positioning structure of claim 17, wherein a single biomaterial is conjugated to the active layer and within the aperture of each of the at least one nanopore.
